(19) United States
(12) Patent Application Publication
(10) Pub. No.: US 2010/0136640 A1
(43) Pub. Date: Jun. 3, 2010

(54) ENHANCED BUTANOL PRODUCING MICROORGANISMS AND METHOD FOR PREPARING BUTANOL USING THE SAME

(75) Inventors: Sang Yup Lee, Daejeon (KR); Jin Hwan Park, Gyeonggi-do (KR); Eleftherios Terry Papoutsakis, Newark, DE (US)

Correspondence Address: MOORE & VAN ALLEN PLLC, P.O. BOX 13706, Research Triangle Park, NC, 27709, US

(73) Assignee: BIOFUELCHEM CO., LTD., Daejeon (KR)

(21) Appl. No.: 12/519,060

(22) PCT Filed: Dec. 14, 2007
(86) PCT No.: PCT/KR07/06525

§ 371 Date: Dec. 22, 2009

Related U.S. Application Data
(60) Provisional application No. 60/875,145, filed on Dec. 15, 2006.
(60) Provisional application No. 60/899,201, filed on Feb. 2, 2007.

(51) Int. Cl. C12P 7/16; C12N 1/21; C12N 1/19; C12N 1/15

(52) U.S. Cl. 435/160; 435/252.3; 435/254.2; 435/254.11; 435/252.33

(57) ABSTRACT

The present invention relates to a recombinant mutant microorganism having enhanced butanol producing capacity and a method for producing butanol using the same. In the microorganism, genes coding for enzymes responsible for the biosynthesis of lactate, ethanol and/or acetate are deleted or attenuated and genes coding for enzymes involved in butanol biosynthesis are introduced and amplified.

### TECHNICAL FIELD

The present invention relates to recombinant mutant microorganisms having enhanced butanol-producing ability in which genes coding for enzymes responsible for the biosynthesis of lactate, ethanol and/or acetate are deleted and genes coding for enzymes involved in butanol biosynthesis are introduced, and a method for producing butanol using the same.

### BACKGROUND ART

With the great increase in oil prices and growing concern about global warming and greenhouse gases, biofuels have recently gained increasing attention with respect to the production thereof using microorganisms. Particularly, biobutanol has an advantage over bioethanol in that it is more highly miscible with fossil fuels thanks to the low oxygen content thereof. Recently emerging as a substitute fuel for gasoline, biobutanol has rapidly increased in market size. The U.S. market for biobutanol amounts to 370 million gal per year, with a price of 3.75 $/gal. Butanol is superior to ethanol as a replacement for petroleum gasoline. With high energy density, low vapor pressure, a gasoline-like octane rating and low impurity content, it can be blended into existing gasoline at much higher proportions than ethanol without compromising performance, mileage, or organic pollution standards. The mass production of butanol by microorganisms can confer economic and environmental advantages of decreasing the import of crude oil and greenhouse gas emissions.

Butanol can be produced through anaerobic ABE (acetone-butanol-ethanol) fermentation by Clostridial strains (Jones, D. T. and Woods, D. R., Microbiol. Rev., 50:484, 1986; Rogers, P., Adv. Appl. Microbiol., 31:1, 1986; Lesnik, E. A. et al., Nucleic Acids Research, 29: 3583, 2001). This biological method was the main technology for the production of butanol and acetone for more than 40 years, until the 1950s. Clostridial strains are difficult to improve further because of complicated growth conditions thereof and the insufficient provision of molecular biology tools and omics technology therefor.

Thus, it is suggested that microorganisms such as E. coli that can grow rapidly under typical conditions and be manipulated using various omics technologies be developed as butanol-producing strains. Particularly, E. coli species, to which little metabolic engineering and omics technology have been applied for the development of butanol-producing strains, have vast potential for development into butanol-producing strains.

Therefore, there is a need for the development of a microorganism having high butanol producing ability, especially a recombinant E. coli, by metabolic engineering such as metabolic network reconstitution by gene deletion, insertion and amplification of desired genes, unlike the prior art wild type Clostridium acetobutylicum.

Recombinant bacteria capable of producing butanol, into which a butanol biosynthesis pathway is introduced, and butanol production using the same have been disclosed (US 2007/0259410 A1; US 2007/0259411 A1), but the production efficiency is modest.

The present inventors have made extensive efforts to develop a microorganism having a high butanol productivity by metabolic engineering, and as a result, constructed a recombinant microorganism by deleting or attenuating genes coding for enzymes involved in the biosynthesis of lactate, ethanol and acetate and introducing or amplifying genes coding for enzymes responsible for butanol biosynthesis, and confirmed that the butanol production was remarkably increased by the recombinant mutant microorganism, thereby completing the present invention.

### SUMMARY OF THE INVENTION

It is a main object of the present invention to provide a recombinant mutant microorganism capable of producing butanol at high efficiency and a preparation method thereof.

It is another object of the present invention to provide a method for producing butanol using the recombinant mutant microorganism.

In order to achieve the above objects, in one aspect, the present invention provides a method for preparing a recombinant mutant microorganism having high butanol productivity, the method comprises: deleting or attenuating at least one selected from the group consisting of genes coding for enzymes involved in lactate biosynthesis, genes coding for enzymes involved in acetate biosynthesis, and genes coding for enzymes involved in ethanol biosynthesis in a microorganism; and introducing or amplifying at least one gene coding for an enzyme involved in butanol biosynthesis into the microorganism.

In another aspect, the present invention provides a recombinant mutant microorganism having high butanol productivity, in which at least one selected from the group consisting of genes coding for enzymes involved in lactate biosynthesis, genes coding for enzymes involved in acetate biosynthesis, and genes coding for enzymes involved in ethanol biosynthesis is deleted or attenuated; and at least one gene coding for an enzyme involved in butanol biosynthesis is introduced or amplified.

In an embodiment of this aspect, a lacI gene (coding for a lac operon repressor) is further deleted in the microorganism so as to enhance the expression of the gene coding for the enzyme involved in butanol biosynthesis.

In another embodiment, the gene coding for enzyme involved in the lactate biosynthesis may be ldhA (coding for lactate dehydrogenase), the gene coding for enzyme involved in the acetate biosynthesis may be pta (coding for phosphoacetyltransferase), and the gene coding for enzyme involved in the ethanol biosynthesis may be adhE (coding for alcohol dehydrogenase).

In the present invention, the enzyme involved in butanol biosynthesis is selected from the group consisting of thiolase (THL), 3-hydroxybutyryl-CoA dehydrogenase (BHBD), crotonase (CRO), butyryl-CoA dehydrogenase (BCD), butyraldehyde dehydrogenase (AAD), butanol dehydrogenase (BDH), and combinations thereof.

In the present invention, the THL may be encoded by a gene selected from the group consisting of thl, thiL, phaA, and atoB, and the BCD may be encoded by a bcd gene derived from Pseudomonas sp., a bcd gene derived from Clostridium sp., and a ydbM gene derived from Bacillus sp. When the gene coding for BCD is a bcd gene derived from Clostridium sp., a chaperone-encoding gene (groESL) and a BCD co-factor-encoding gene (etfAB) are further introduced into the microorganism. Also, the gene coding for the BHBD may be a hbd gene derived from Clostridium sp. or a paaH gene derived from E. coli. The gene coding for the CRO may be a crt gene derived from Clostridium sp. or a paaFG gene derived from E. coli. The gene coding for the AAD may be an adhE gene derived from Clostridium sp. or a mhpF gene derived from E. coli.

In the present invention, the gene coding for the enzyme involved in the butanol biosynthesis may be introduced into the host cell by an expression vector containing a strong promoter. This strong promoter may be selected from the group consisting of a trc promoter, a tac promoter, a T7 promoter, a lac promoter and a trp promoter. The expression vector containing the strong promoter may further contain a gene coding for an enzyme selected from the group consisting of 3-hydroxybutyryl-CoA dehydrogenase, thiolase, butyraldehyde dehydrogenase, crotonase, butanol dehydrogenase, butyryl-CoA dehydrogenase and combinations thereof.

In still another aspect, the present invention provides a recombinant mutant microorganism having high butanol productivity, in which genes coding for enzymes involved in lactate biosynthesis, genes coding for enzymes involved in acetate biosynthesis, and genes coding for enzymes involved in ethanol biosynthesis are deleted or attenuated; and genes coding for thiolase (THL), 3-hydroxybutyryl-CoA dehydrogenase (BHBD), crotonase (CRO), butyryl-CoA dehydrogenase (BCD), butyraldehyde dehydrogenase (AAD), butanol dehydrogenase (BDH), a chaperone protein (groESL), and BCD co-factors (etfAB) are introduced or amplified.

In further still another aspect, the present invention provides a method for producing butanol, the method comprises: culturing the recombinant mutant microorganism to produce butanol; and recovering the butanol from the culture broth.

Other features, advantages, and embodiments of the present invention will be obvious from the following detailed description and the accompanying claims.

### BRIEF DESCRIPTION OF DRAWINGS

FIG. 1 is a diagram showing a butanol biosynthesis pathway in Clostridium acetobutylicum;

FIG. 2 shows a construction process and a genetic map of pKKhbdadhEthiL (pKKHAT) vector.

FIG. 3 shows a construction process and a genetic map of pKKhbdadhEatoB (pKKHAA) vector.

FIG. 4 shows a construction process and a genetic map of pKKhbdadhEphaA (pKKHAP) vector.

FIG. 5 shows a construction process and a genetic map of pKKhbdydbMadhEphaA (pKKHYAP) vector.

FIG. 6 shows a construction process and a genetic map of pKKhbdbcdPA01adhEphaA (pKKHPAP) vector.

FIG. 7 shows a construction process and a genetic map of pKKhbdbcdKT2440adhEphaA (pKKHKAP) vector.

FIG. 8 shows a construction process and a genetic map of pKKhbdgroESLadhEphaA (pKKHGAP) vector.

FIG. 9 shows a construction process and a genetic map of pTrc184bcdbdhABcrt (pTrc184BBC) vector.

FIG. 10 shows a butanol biosynthesis pathway in the case where a part of genes derived from C. acetobutylicum involved in a butanol biosynthesis pathway, was substituted by genes derived from E. coli.

FIG. 11 shows a construction process and a genetic map of pKKmhpFpaaFGHatoB (pKKMPA) vector.

FIG. 12 shows a construction process and a genetic map of pTrc184bcdetfABbdhABgroESL (pTrc184BEBG) vector.

### DETAILED DESCRIPTION OF THE INVENTION, AND PREFERRED EMBODIMENTS

The term “deletion”, as used herein in relation to a gene, means that the gene cannot be expressed or, if it is expressed, cannot lead to enzyme activity, due to the mutation, substitution, deletion or insertion of any number of nucleotides from a single base to an entire piece of the gene, resulting in the blockage of the biosynthesis pathway in which an enzyme encoded by gene is involved.

By the term “attenuation”, as used herein in relation to a gene, it is meant that the activity of the enzyme expressed by the gene is decreased by the mutation, substitution, deletion, or insertion of any number of nucleotides, ranging from a single base to entire pieces of the gene, resulting in the blockage of a part or a critical part of the biosynthesis pathway in which an enzyme encoded by gene is involved.

The term “amplification”, as used herein in relation to a gene, is intended to refer to an increase in the activity of the enzyme corresponding to the gene due to the mutation, substitution, deletion or insertion of any number of nucleotides from a single base to partial pieces of the gene, or by the introduction of an exogenous gene coding for the same enzyme.

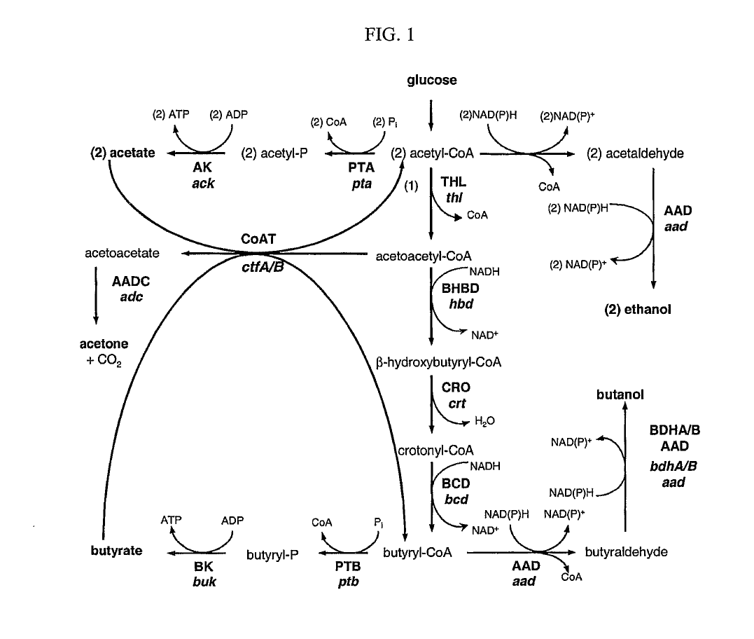

The present invention employs the butanol biosynthesis pathway of Clostridium acetobutylicum as a model for producing butanol in the recombinant microorganism (FIG. 1). When account is taken of both the pathway of FIG. 1 and the pathway of E. coli, enzymes including thiolase (THL), 3-hydroxybutyryl-CoA dehydrogenase (BHBD), crotonase (CRO), butyryl-CoA dehydrogenase (BCD), butyraldehyde dehydrogenase (AAD) and butanol dehydrogenase (BDH) are believed to be involved in the biosynthesis of butanol.

The gene thl derived from Clostridium sp. has already been identified to effectively express THL in E. coli (Bermejo, L. L. et al., Appl. Environ. Microbiol., 64:1079, 1998). In addition to thl, the gene thiL is known to encode THL in Clostridium sp. (Nolling, J. et al., J. Bacteriol., 183:4823, 2001). THL functions to convert acetyl-CoA into acetoacetyl-CoA. In an example of the present invention, phaA derived from Ralstonia sp. or atoB derived from E. coli was identified to perform the same function as thl or thiL. Accordingly, as long as it is expressed to show THL activity in the host cells, any gene coding for THL, even if exogenous, can be used without limitations.

Also, Bennett et al. reported that among enzymes necessary for the production of butyryl-CoA from acetoacetyl-CoA, BHBD and CRO except for BCD are expressed in E. coli (Boynton, Z. L. et al., J. Bacteriol., 178:3015, 1996). Accordingly, hbd and crt, both of which are derived from Clostridium sp., are introduced as genes encoding BHBD and CRO, respectively, in the recombinant microorganism according to the present invention. Both the genes, although exogenous, can be used without limitations, as long as they are expressed and show the same activity in the host cells. In an example of the present invention, butanol was also produced even when hbd and crt derived from Clostridium sp. were substituted respectively with paaH (gene coding for 3-hydroxy-acyl-CoA dehydrogenase) and paaFG (a gene coding for enoyl-CoA hydratase) derived from E. coli.

According to the article, however, it is reported that E. coli has no BCD function because of the poor expression of BCD or its cofactors (electron transfer flavoproteins putatively coded by the Clostridium acetobutylicum genes (etfB and etfA)) therein, or no in vitro activity is observed because of the poor stability of BCD or its cofactors.

In accordance with the present invention, low-level expression of butyryl-CoA dehydrogenase can be overcome by the introduction of bcd derived from Pseudomonas aeruginosa or Pseudomonas putida, or ydbM derived from Bacillus subtilis. As long as it is expressed to show BCD activity in the host cells, a BCD gene, even though exogenous, can be used without limitations.

In an alternative embodiment, bcd derived from Clostridium acetobutylicum may be introduced together with a chaperone-encoding gene (groESL), so as to solve the problem of low-level expression of butyryl-CoA dehydrogenase. When the bcd of Clostridium acetobutylicum and the chaperone-encoding gene (groESL) was introduced into E. coli host cells, butanol productivity thereof is increased as demonstrated in the example of the present invention. In addition to the bcd derived from Clostridium acetobutylicum and the chaperone-encoding gene (groESL), the introduction of a gene coding for BCD cofactors was found to significantly increase butanol production capacity, as demonstrated in an example of the present invention.

Previously, the present inventors reported that although a gene coding for BDH is not introduced, a host cell (e.g., E. coli) which harbors a gene encoding an enzyme (AdhE, which converts butyryl-CoA to butanol), can produce butanol using butyryl-CoA serving as an intermediate.

In the present invention, the bdhAB derived from Clostridium sp. is introduced as a BDH-encoding gene in order to improve the yield of conversion from butyryl-CoA to butanol. BDH encoding genes derived from microorganisms other than bdhAB derived from Clostridium sp. may be used without limitations as long as they are expressed to show the same BDH activity.

Improvement in conversion from butyryl-CoA to butanol can be brought about by introducing the AAD-encoding gene, adhE, derived from Clostridium sp., in accordance with the present invention. ADD-encoding genes derived from microorganisms other than adhE derived from Clostridium sp. can be used without limitations as long as they are expressed to show the same AAD activity. It is well known that mhpF derived from E. coli encodes acetaldehyde dehydrogenase (Ferrandez, A. et al., J. Bacteriol., 179:2573, 1997). When adhE derived from Clostridium sp. is substituted with mhpF (coding for acetaldehyde dehydrogenase or butyraldehyde) derived from E. coli, butanol can be produced, as demonstrated in an example of the present invention.

In consideration of the pathway of FIG. 1 and the pathway of E. coli, it is understood that butanol production can be improved by shutting the biosynthesis pathways for acetate, ethanol and lactate, which compete with the butanol biosynthesis pathway. These competing pathways are shut down in the host cells of interest before introducing genes involved in the butanol biosynthesis pathway in accordance with the present invention.

In practice, genes coding for enzymes responsible for the biosynthesis of lactate, acetate and/or ethanol in E. coli wild-type W3110 are attenuated or deleted so as to construct a mutant E. coli which has enhanced butanol productivity in accordance with the present invention.

In detail, ldhA (coding for lactate dehydrogenase which is involved in the biosynthesis of lactate), pta (coding for phosphoacetyltransferase which is involved in the biosynthesis of acetate) and adh (coding for alcohol dehydrogenase which is involved in the biosynthesis of ethanol) are deleted. It should be understood that as long as the competing biosynthesis pathways can be shot down, the deletion of genes other than these genes is within the scope of the present invention.

Afterwards, lacI (coding for lac operon repressor) was additionally deleted, so as to increase the expression level of the genes encoding the enzymes responsible for butanol biosynthesis.

In greater detail, E. coli WLLPA, which lacks the three genes (lahA, pta and adh) plus lacI, and E. coli WLL, which lacks ldhA and lacI, were constructed, followed by introducing genes encoding the enzymes responsible for butanol biosynthesis, including THL, BHBD, CRO, BCD, cofactors of BCD, and BHD, thereinto, thus constructing recombinant mutant microorganisms having excellent butanol productivity.

The THL-encoding gene, the BHBD-encoding gene, the CRO-encoding gene, the

BCD-encoding gene, the BCD cofactor-encoding gene, the AAD-encoding gene and the BDH-encoding gene may be introduced into a host cell by an expression vector containing a strong promoter. Examples of the strong promoter useful in the present invention include, but are not limited to, a trc promoter, a tac promoter, a T7 promoter, a lac promoter and a trp promoter.

Finally, the genes encoding thiolase (THL), 3-hydroxybutyryl-CoA dehydrogenase (BHBD), crotonase (CRO), butyryl-CoA dehydrogenase (BCD), butyraldehyde dehydrogenase (AAD), butanol dehydrogenase (BDH), a chaperone protein (groESL) and BCD cofactors (etfAB) are introduced into E. coli strains, in which genes encoding the enzymes involved in the biosynthesis of lactate, acetate, and/or ethanol are attenuated or deleted, to prepare recombinant mutant E. coli, thus confirming that butanol productivity is remarkably improved in the recombinant mutant E. coli.

### Examples

A better understanding of the present invention may be obtained through the following examples which are set forth to illustrate, but are not to be construed as the limit of the present invention.

Although, in the following examples, E. coli W3110 was used as a host microorganism, it will be obvious to those skilled in the art that other E. coli strains, bacteria, yeasts and fungi can also be used as host cells by deleting target gene to be deleted and introducing genes involved in butanol biosynthesis, in order to produce butanol.

Further, although genes derived from a specific strain are exemplified as target genes to be introduced in the following examples, it is obvious to those skilled in the art that as long as they are expressed to show the same activity in the host cells, any genes may be employed without limitations.

Also, it should be noted that although only specific culture media and methods are exemplified in the following example, saccharified liquid, such as whey, CSL (corn steep liquor), etc, and the other media, and various culture methods, such as fed-batch culture, continuous culture, etc. (Lee et al., Bioprocess Biosyst. Eng., 26:63, 2003; Lee et al., Appl. Microbiol. Biotechnol., 58:663, 2002; Lee et al., Biotechnol. Lett., 25:111, 2003; Lee et al., Appl. Microbiol. Biotechnol., 54:23, 2000; Lee et al., Biotechnol. Bioeng., 72:41, 2001) also fall within the scope of the present invention.

### Example 1

### Preparation of Recombinant Mutant Microorganism Having High Butanol Productivity

### 1-1: Deletion of lacI Gene

In E. coli W3110 (ATTC 39936), the lacI gene coding for the lac operon repressor, which functions to inhibit the transcription of an lac operon required for the metabolism of lactose, was deleted through one-step inactivation (Warner et al., PNAS, 6:97(12):6640, 2000) using primers of SEQ ID NOS: 1 and 2, thus removing antibiotic resistance from the bacteria.

[SEQ ID NO: 1]

lacI_1stup:
5′-gtgaaaccagtaacgttatacgatgtcgcagagtatgccgg

[SEQ ID NO: 2]

lacI_1stdo:
5′-tcactgcccgctttccagtcgggaaacctgtcgtgccagctg
cattaatgcacttaacggctgacatggg-3′

### 1-2: Deletion of ldhA, pta and adhE Genes

In the lacI-knockout E. coli W3110 of Example 1-1, ldhA (coding for lactate dehydrogenase), pta (coding for phosphotransacetylase) and adhE (coding for alcohol dehydrogenase) were further deleted by one-step inactivation using primers of SEQ ID NOS: 3 to 8.

That is, the three genes were deleted from E. coli W3110 competent cells lacking lacI, prepared in Example 1-1, thus constructed a novel mutant WLLPA strain.

Additionally, ldhA (coding for lactate dehydrogenase) was deleted in the lacI-knockout E. coli W3110 competent cells of Example 1-1 through the one step inactivation with the aid of primers of SEQ ID NOS: 3 and 4, thus constructed a novel mutant WLL strain.

[SEQ ID NO: 3]

1dhA 1stup:
5′-atgaaactcgccgtttatagcacaaaacagtacgacaagaag
tacctgcagattgcagcattacacgtcttg-3′

[SEQ ID NO: 4]

ldhA 1stdo:
5′-ttaaaccagttcgttcgggcaggtttcgcctttttccagattgct
taagtcacttaacggctgacatggga-3′

[SEQ ID NO: 5]

pta 1stup:
5′-gtgtcccgtattattatgctgatccctaccggaaccagcgtcggtc
tgacgattgcagcattacacgtcttg-3′

[SEQ ID NO: 6]

pta 1stdo:
5′-ttactgctgctgtgcagactgaatcgcagtcagcgcgatggtgta
gacgaacttaacggctgacatggg-3′

[SEQ ID NO: 7]

adhE 1stup:
5′-cgtgaatatgccagtttcactcaagagcaagtagacaaaatctt
ccgcgcgattgcagcattacacgtcttg-3′

[SEQ ID NO: 8]

adhE 1stdo:
5′-taatcacgaccgtagtaggtatccagcagaatctgtttcagctc
ggagatcacttaacggctgacatggg-3′

### 1-3: Construction of pKKhbdadhEthiL (pKKHAT) Vector

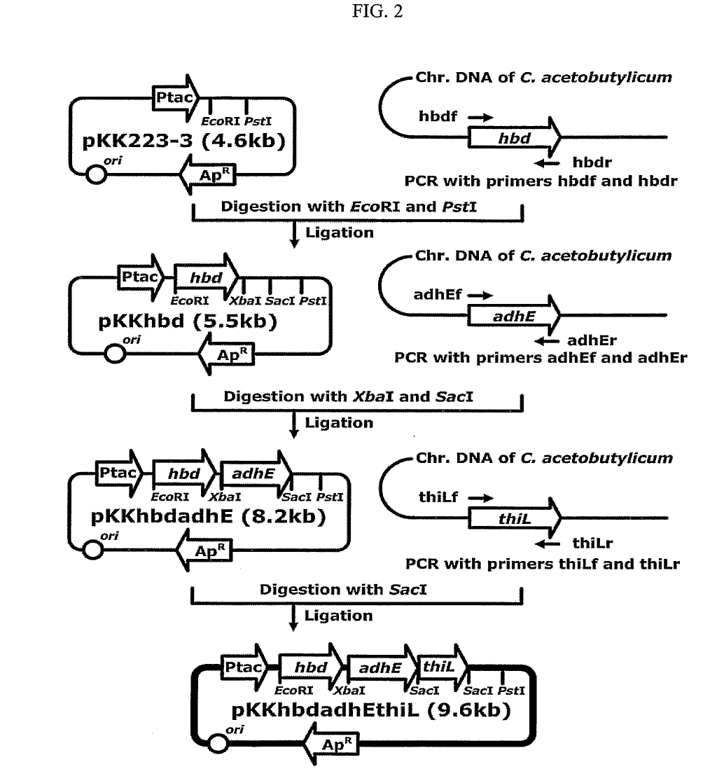

Genes necessary for the butanol biosynthesis pathway, including hbd (coding for 3-hydroxybutyryl-CoA dehydrogenase), adhE (coding for butyraldehyde dehydrogenase: the same spell, but different in function from the adhE (coding for alcohol dehydrogenase) of 1-2) and thiL (coding for thiolase) was amplified using primers of SEQ ID NOS: 9 to 14 with the chromosomal DNA of Clostridium acetobutylicum (KCTC 1724) serving as a template, and they were sequentially cloned into a pKK223-3 expression vector (Pharmacia Biotech), thus constructed a recombinant expression vector, named pKKhbdadhEthiL (pKKHAT) (FIG. 2).

[SEQ ID NO: 9]

hbdf:
5′-acgcgaattcatgaaaaaggtatgtgttat-3′

[SEQ ID NO: 10]

hbdr:
5′-gcgtctgcaggagctcctgtctctagaatttgataatggggattct
t-3′

[SEQ ID NO: 11]

adhEf:
5′-acgctctagatataaggcatcaaagtgtgt-3′

[SEQ ID NO: 12]

adhEr:
5′-gcgtgagctccatgaagctaatataatgaa-3′

[SEQ ID NO: 13]

thiLf:
5′-acgcgagctctatagaattggtaaggatat-3′

[SEQ ID NO: 14]

thiLr:
5′-gcgtgagctcattgaacctccttaataact-3′

### 1-4: Construction of pKKhbdadhEatoB (pKKHAA) Vector

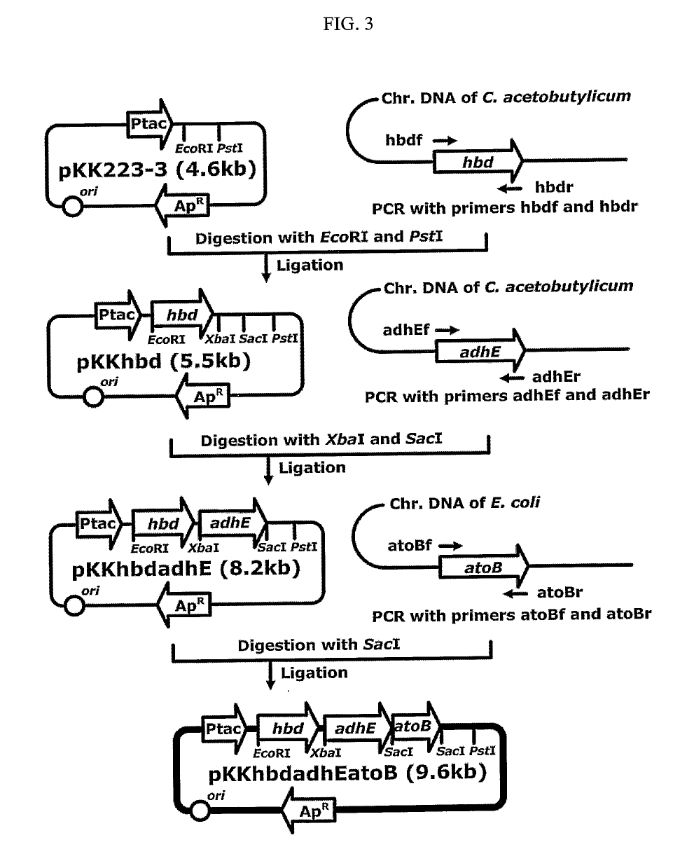

To clone the atoB (coding for acetyl-CoA acetyltransferase) of Escherichia coli W3110 into the pKKhbdadhE vector (FIG. 2), PCR was performed on the chromosomal DNA of Escherichia coli W3110 using primers of SEQ ID NOS: 15 and 16, with 24 cycles of denaturing at 95° C. for 20 sec, annealing at 55° C. for 30 sec and extending at 72° C. for 90 sec. The PCR product (atoB) obtained was digested with SacI and inserted into the pKKhbdadhE vector digested with the same restriction enzyme (SacI), thus constructed a novel recombinant vector, named pKKhbdadhEatoB (pKKHAA) (FIG. 3).

[SEQ ID NO: 15]

| | |
|---|---|
| atof: | 5′-atacgagctctacggcgagcaatggatgaa-3′ |

[SEQ ID NO: 16]

| | |
|---|---|
| ator: | 5′-gtacgagctcgattaattcaaccgttcaat-3′ |

### 1-5: Construction of pKKhbdadhEphaA (pKKHAP) Vector

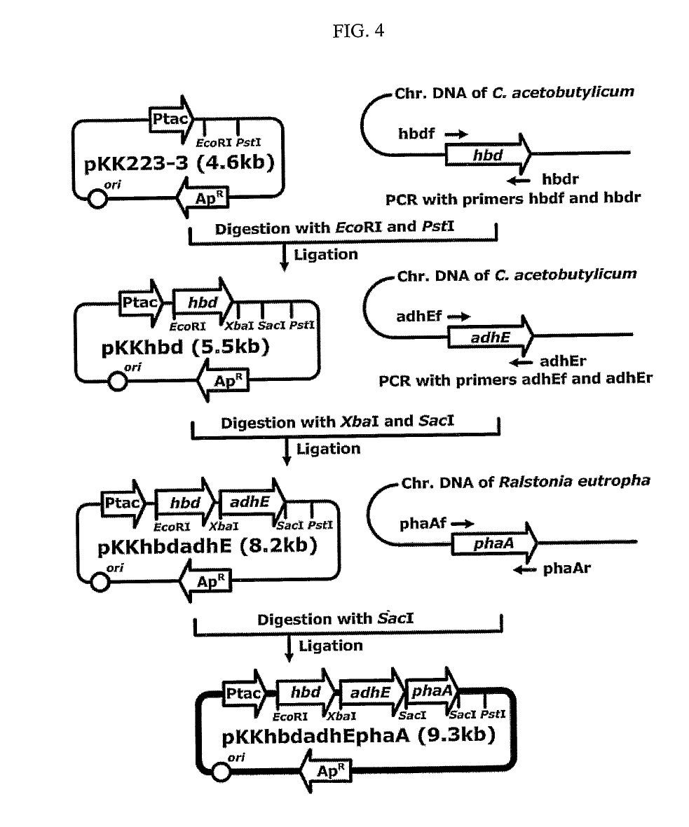

To clone the phaA (coding for thiolase) of Ralstonia eutropha (KCTC 1006) into the pKKhbdadhE vector, PCR was performed using primers of SEQ ID NOS: 17 and 18, with the chromosomal DNA of Ralstonia eutropha serving as a template. The PCR product (phaA) obtained was cleaved with SacI and inserted into the pKKhbdadhE vector digested with the same restriction enzyme (SacI), thus constructed a novel recombinant vector, named pKKhbdadhEphaA (pKKHAP) (FIG. 4).

[SEQ ID NO: 17]

phaAf:
5′-agtcgagctcaggaaacagatgactgacgttgtcatcgt-3′

[SEQ ID NO: 18]

phaAr:
5′-atgcgagctcttatttgcgctcgactgcca-3′

### 1-6: Construction of pKKhbdydbMadhEphaA (pKKHYAP) Vector

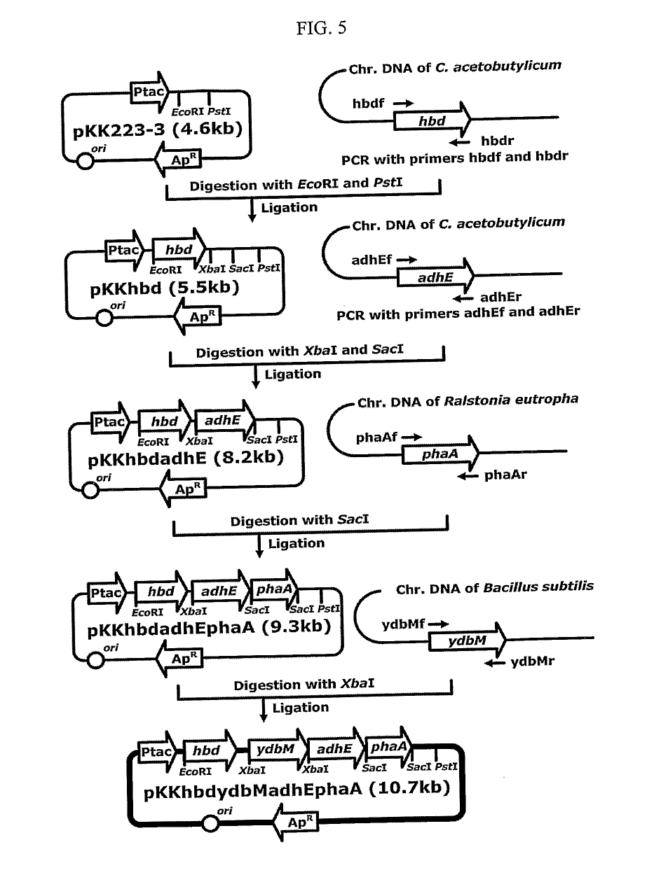

To clone the ydbM (coding for hypothetical protein) of Bacillus subtilis (KCTC 1022) into the pKKhbdadhE vector, PCR was performed using primers of SEQ ID NOS: 19 and 20 with the chromosomal DNA of Bacillus subtilis serving as a template. The PCR product (ydbM) obtained was cleaved with XbaI and inserted into the pKKhbdadhEphaA vector digested with the same restriction enzyme (XbaI), thus constructed a novel recombinant vector, named pKKhbdydbMadhEphaA (pKKHYAP) (FIG. 5).

[SEQ ID NO: 19]

| | |
|---|---|
| ydbMf: | 5′-agcttctagagatgggttacctgacatata-3′ |

[SEQ ID NO: 20]

| | |
|---|---|
| ydbMr: | 5′-agtctctagattatgactcaaacgcttcag-3′ |

### 1-7: Construction of pKKhbdbcdPA01adhEphaA (pKKHPAP) Vector

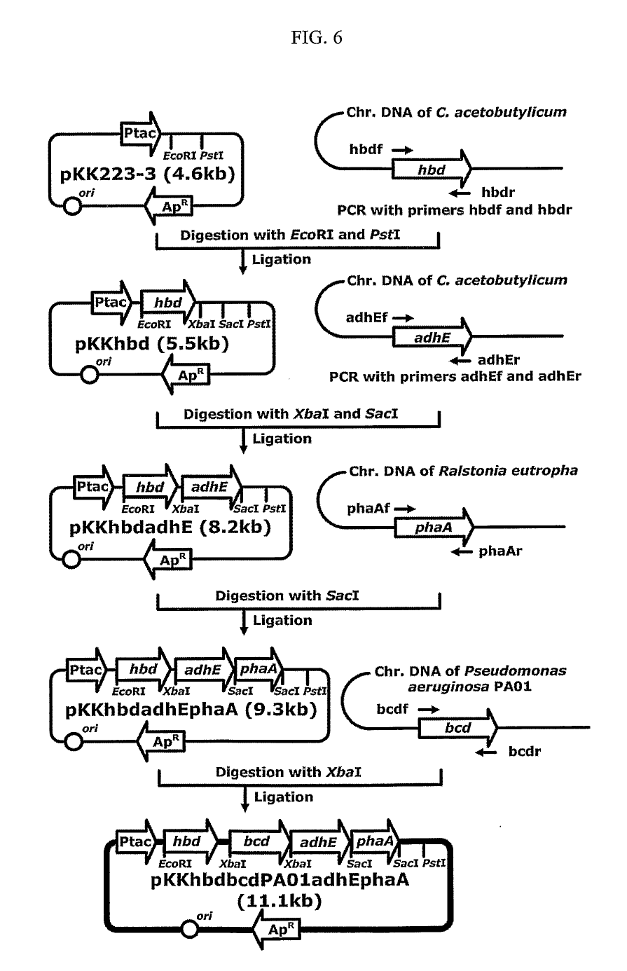

To clone the bcd (coding for butyryl-CoA dehydrogenase) of Pseudomonas aeruginosa PA01 (KCTC 1637) into the pKKhbdadhEphaA vector, PCR was performed using primers of SEQ ID NOS: 21 and 22 with the chromosomal DNA of Pseudomonas aeruginosa PA01 serving as a template. The PCR product (bcd) obtained was cleaved with XbaI and inserted into the pKKhbdadhEphaA (pKKHAP) vector digested with the same restriction enzyme (XbaI), thus constructed a novel recombinant vector, named pKKhbdbcdPA01adhEphaA (pKKHPAP) (FIG. 6).

[SEQ ID NO: 21]

| | |
|---|---|
| bcdPA01f: | 5′-agcttctagaactgctccttggacagcgcc-3′ |

[SEQ ID NO: 22]

| | |
|---|---|
| bcdPA01r: | 5′-agtctctagaggcaggcaggatcagaacca-3′ |

### 1-8: Construction of pKKhbdbcdKT2440adhEphaA (pKKHKAP) Vector

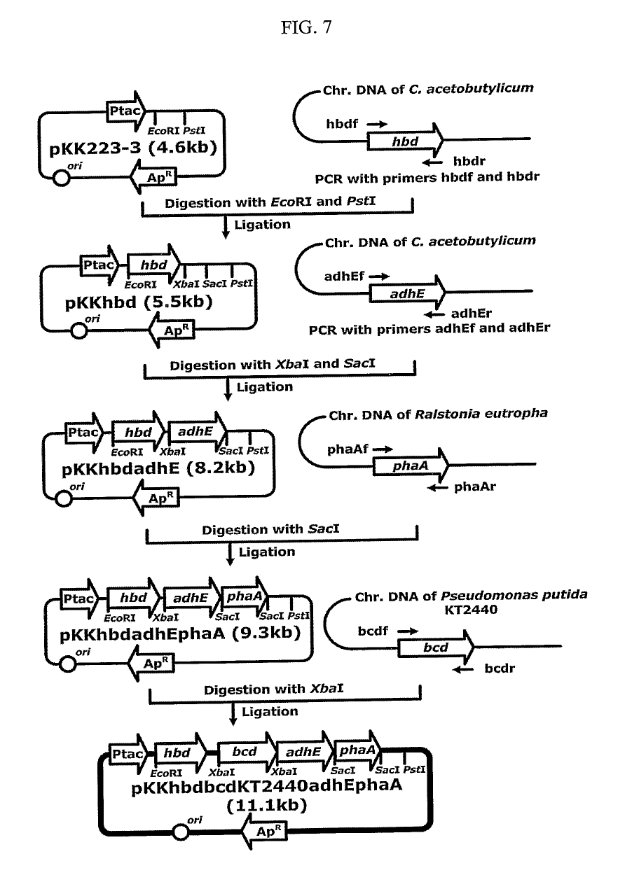

To clone the bcd (coding for butyryl-CoA dehydrogenase) of Pseudomonas putida KT2440 (KCTC 1134) into the pKKhbdadhEphaA vector, PCR was performed using primers of SEQ ID NOS: 23 and 24 with the chromosomal DNA of Pseudomonas putida KT2440 serving as a template. The PCR product (bcd) obtained was cleaved with XbaI and inserted into the pKKhbdadhEphaA vector digested with the same restriction enzyme (XbaI), thus constructed a novel recombinant vector, named pKKhbdbcdKT2440adhEphaA (pKKHKAP) (FIG. 7).

[SEQ ID NO: 23]

| | |
|---|---|
| bcdKT2440f: | 5′-agcttctagaactgttccttggacagcgcc-3′ |

[SEQ ID NO: 24]

| | |
|---|---|
| bcdKT2440r: | 5′-agtctctagaggcaggcaggatcagaacca-3′ |

### 1-9: Construction of pKKhbdgroESLadhEphaA (pKKHGAP) Vector

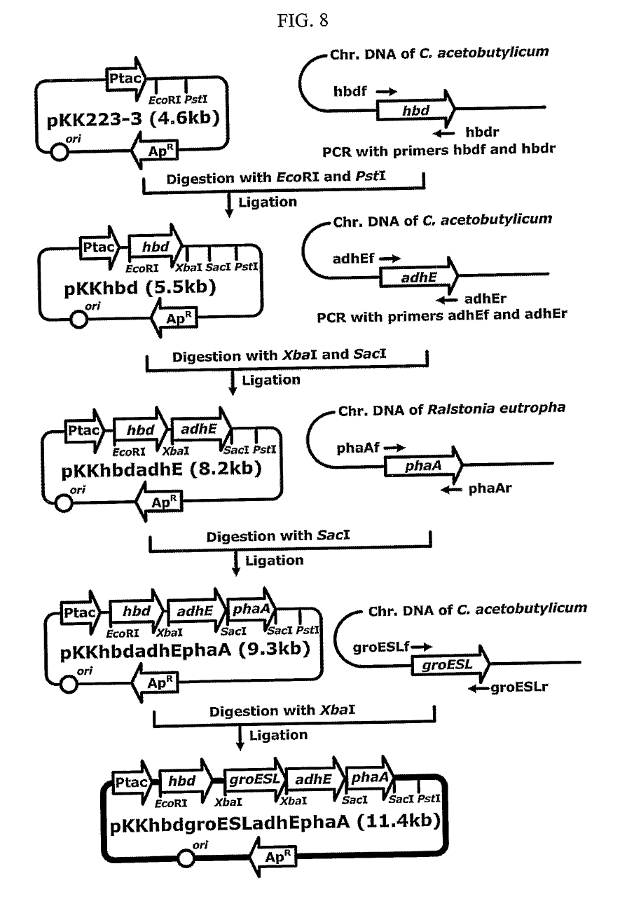

PCR was performed using primers of SEQ ID NOS: 25 and 26 with the chromosomal DNA of Clostridium acetobutylicum serving as a template. The PCR product (groESL) obtained was cleaved with XbaI and inserted into the pKKhbdadhEphaA vector digested with the same restriction enzyme (XbaI), thus constructed a novel recombinant vector, named pKKhbdgroESLadhEphaA (pKKHGAP) (FIG. 8).

[SEQ ID NO: 25]

| | |
|---|---|
| groESLf: | 5′-agcttctagactcaagattaacgagtgcta-3′ |

[SEQ ID NO: 26]

| | |
|---|---|
| groESLr: | 5′-tagctctagattagtacattccgcccattc-3′ |

### 1-10: Construction of pTrc184bcdbdhABcrt Vector

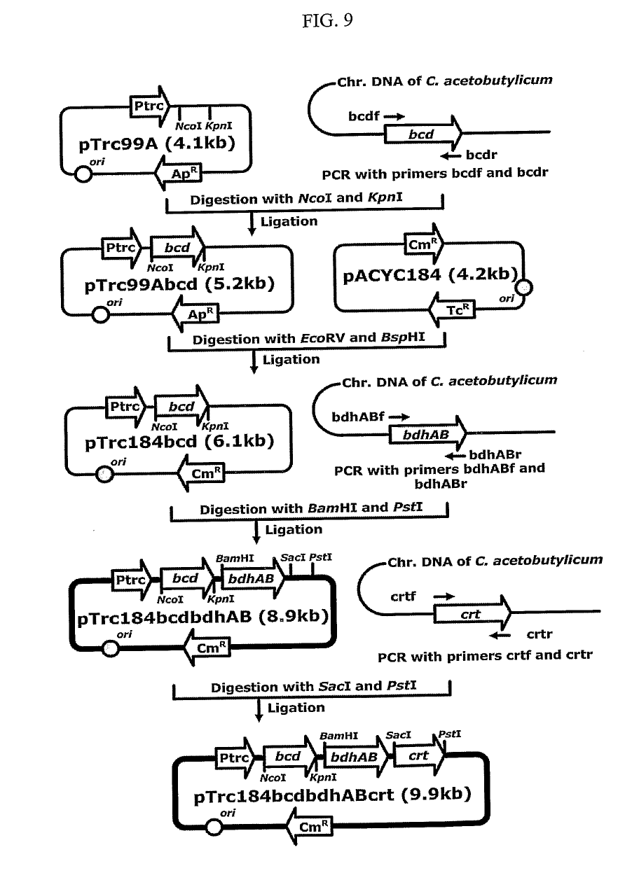

PCR was performed using primers of SEQ ID NOS: 27 and 28, with the chromosomal DNA of Clostridium acetobutylicum serving as a template. The PCR product (bcd) obtained was digested with NcoI and KpnI and cloned into a pTrc99A expression vector (Amersham Pharmacia Biotech), thus constructed a recombinant vector named pTrc99Abcd. After the pTrc99Abcd vector was digested with BspHI and EcoRV, the DNA fragment thus excised was inserted into pACYC184 (New England Biolabs) which was previously treated with the same restriction enzymes (BspHI and EcoRV), thus constructed a recombinant expression vector for expressing the bcd gene, named pTrc184bcd (FIG. 9).

[SEQ ID NO: 27]

| | |
|---|---|
| bcdf: | 5′-agcgccatggattttaatttaacaag-3′ |

[SEQ ID NO: 28]

| | |
|---|---|
| bcdr: | 5′-agtcggtacccctccttaaattatctaaaa-3′ |

PCR was performed using primers of SEQ ID NOS: 29 and 30, with the chromosomal DNA of Clostridium acetobutylicum serving as a template. The PCR product (bdhAB) obtained was digested with BamHI and PstI and inserted into the pTrc184bcd expression vector digested with the same restriction enzymes (BamHI and PstI), thus constructed a recombinant vector, named pTrc184bcdbdhAB (pTrc184BB), which contain both bcd and bdhAB.

[SEQ ID NO: 29]

bdhABf:
5′-acgcggatccgtagtttgcatgaaatttcg-3′

[SEQ ID NO: 30]

bdhABr:
5′-agtcctgcagctatcgagctctataatggctacgcccaaac-3′

PCR was performed using primers of SEQ ID NOS: 31 and 32, with the chromosomal DNA of Clostridium acetobutylicum serving as a template. The PCR product (crt) obtained was digested with SacI and PstI and inserted into the pTrc184bcdbdhAB vector digested with the same restriction enzymes (SacI and PstI), thus constructed a recombinant vector, named pTrc184bcdbdhABcrt (pTrc184BBC), which contain all of the bcd gene, the bdhAB gene and the crt gene (FIG. 9).

[SEQ ID NO: 31]

| | |
|---|---|
| crtf: | 5′-actcgagctcaaaagccgagattagtacgg-3′ |

[SEQ ID NO: 32]

| | |
|---|---|
| crtr: | 5′-gcgtctgcagcctatctatttttgaagcct-3′ |

### 1-11: Preparation of Butanol-Producing Microorganisms

E. coli W3110 (WLLPA) lacking lacI, ldhA, pta and adhE and E. coli W3110 (WLL) lacking lacI and ldhA, respectively prepared in Examples 1-1 and 1-2, were transformed with the pTrc184bcdbdhABcrt (pTrc184BBC) vector of Example 1-10 and the vector selected from the group consisting of pKKhbdadhEthiL (pKKHAT), pKKhbdadhEatoB (pKKHAA), pKKhbdydbMadhEphaA (pKKHYAP), pKKhbdadhEphaA (pKKHAP), pKKhbdbcdPA01adhEphaA (pKKHPAP), pKKhbdbcdKT2440adhEphaA (pKKHKAP) and pKKhbdgroESLadhEphaA (pKKHGAP) constructed in Examples 1-3 to 1-9, thus prepared recombinant mutant microorganisms (WLLPA+pKKHPAP+pTrc184BBC, WLL+pKKHAT+pTrc184BBC, WLL+pKKHAA+pTrc184BBC, WLL+pKKHAP+pTrc184BBC, WLL+pKKHYAP+pTrc184BBC, WLL+pKKHPAP+pTrc184BBC, WLL+pKKHKAP+pTrc184BBC, and WLL+pKKHGAP+pTrc184BBC) capable of producing butanol.

### 1-12: Assay for Butanol Productivity

The butanol-producing microorganisms prepared in Example 1-11 were selected on LB plates containing 50 μg/ml ampicillin and 30 μg/ml chloramphenicol. For the selection of the WLLPA+pKKHPAP+pTrc184BBC strain, kanamycin was added in an amount of 30 μg/ml to the LB plates. The recombinants were precultured at 37° C. for 12 hr in 10 ml of LB broth. After being autoclaved, 100 mL of LB broth maintained at 80° C. or higher in a 250 mL flask was added with glucose (5 g/L) and cooled to room temperature in an anaerobic chamber purged with nitrogen gas. 2 mL of the preculture was inoculated into the flask and cultured at 37° C. for 10 hr. Then, 2.0 liters of a medium containing 20 g of glucose, 2 g of KH₂PO₄, 15 g of (NH₄)₂SO₄.7H₂O, 20 mg of MnSO₄.5H₂O, 2 g of MgSO₄.7H₂O, 3 g of yeast extract, and 5 ml of a trace metal solution (10 g FeSO₄.7H₂O, 1.35 g CaCl₂, 2.25 g ZnSO₄.7H₂O, 0.5 g MnSO₄.4H₂O, 1 g CuSO₄.5H₂O, 0.106 g (NH₄)₆Mo₇O₂₄.4H₂O, 0.23 g Na₂B₄O₇.10H₂O, and 35% HCl 10 ml per liter of distilled water) per liter of distilled water in a 5 L fermenter (LiFlus GX, Biotron Inc., Korea) was autoclaved and cooled from 80° C. or higher to room temperature with nitrogen supplied at a rate of 0.5 vvm for 10 hr. In the fermenter, the culture was carried out at 37° C., 200 rpm with shaking at 200 rpm. During the cultivation, pH to be maintained at 6.8 by automatic feeding with 25% (v/v) NH₄OH and nitrogen gas was supplied at a rate of 0.2 vvm (air volume/working volume/minute).

When the glucose of the medium was completely exhausted, as measured using a glucose analyzer (STAT, Yellow Springs Instrument, Yellow Springs, Ohio, USA), the medium was analyzed for butanol concentration using gas chromatography (Agillent 6890N GC System, Agilent Technologies Inc., CA, USA) equipped with a packed column (Supelco Carbopack™ B AW/6.6% PEG 20M, 2 m×2 mm ID, Bellefonte, Pa., USA).

As a result, as shown in Table 1, wild-type E. coli W3110 did not produce butanol, whereas it was produced from the recombinant mutant microorganisms according to the present invention. In addition, all of the genes encoding thiolase (thiL, phaA, atoB) were observed to show activities. Particularly, the butyryl-CoA dehydrogenase of Pseudomonas aeruginosa or Pseudomonas putida is superior to that of Clostridium acetobutylicum in terms of activity, as demonstrated by butanol productivity.

TABLE 1

| | | Butanol |
|---|---|---|
| Strains | Containing genes | (mg/L) |
| W3110 | — | ND¹ |
| WLL + pKKHAT + pTrc184BBC | hbd, adhE, thiL, bcd, bdhAB, crt | 1.2 |
| WLL + pKKHAA + pTrc184BBC | hbd, adhE, atoB, bcd, bdhAB, crt | 1.3 |
| WLL + pKKHAP + pTrc184BBC | hbd, adhE, phaA, bcd, bdhAB, crt | 1.4 |
| WLL + pKKHYAP + pTrc184BBC | hbd, adhE, ydbM, phaA, bcd, bdhAB, crt | 1.7 |
| WLL + pKKHPAP + pTrc184BBC | hbd, adhE, bcdPA01, phaA, bcd, bdhAB, | 3.1 |
| WLLPA + pKKHPAP + pTrc184BBC | crt | 4.5 |
| WLL + pKKHKAP + pTrc184BBC | Hbd, adhE, bcdKT2440, phaA, bcd, | 9.1 |
| | bdhAB, crt | |
| WLL + pKKHGAP + pTrc184BBC | hbd, adhE, groESL, phaA, bcd, bdhAB, | 13.5 |
| | crt | |

¹Not detected.

Also, the butanol productivity was greatly increased by the co-introduction of the chaperone-encoding gene (groESL) and the bcd derived from Clostridium acetobutylicum (WLL+pKKHGAP+pTrc184BBC). Accordingly, the chaperone protein is found to greatly promote the activity of butyryl-CoA dehydrogenase, as demonstrated from the fact that when groESL was introduced, together with the bcd derived from Clostridium acetobutylicum, the butanol productivity increased more that 10-fold.

Previously, the present inventors reported that when the recombinant E. coli into which genes responsible for butanol biosynthesis were introduced, the E. coli strain in which only lacI was deleted could produce butanol. As is apparent from the data of Table 1, butanol production is further increased when ldhA in addition to lacI is deleted. Moreover, the additional deletion of pta and adhE was shown to further improve the butanol productivity. Taken together, the data obtained above demonstrate that the blockage of the lactate biosynthesis pathway, the acetate biosynthesis pathway and/or the ethanol biosynthesis pathway, all of which compete with the butanol biosynthesis pathway, makes a contribution to butanol production.

### Example 2

### Production of Butanol from Recombinant Microorganisms Introduced with Genes Derived from E. coli and C. acetobutylicum

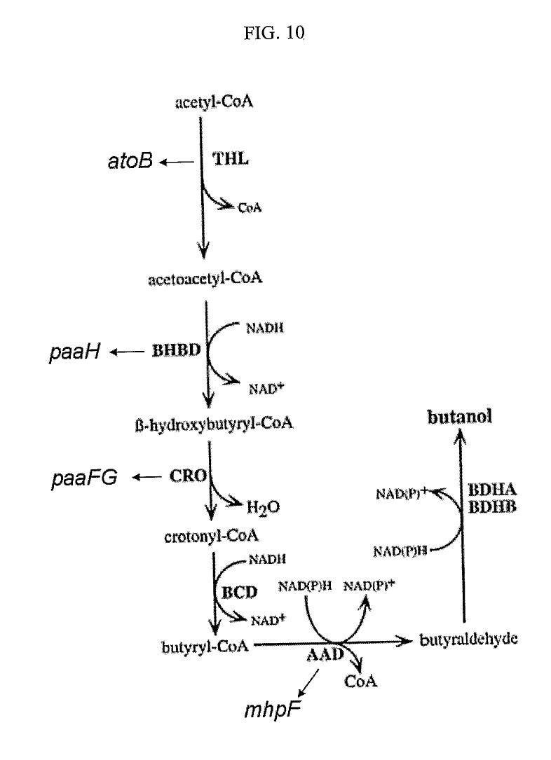

In this example, when the genes derived from C. acetobutylicum, responsible for the butanol biosynthesis pathway, were partially substituted with genes derived from E. coli, butanol productivity was measured (FIG. 10). In detail, when adhE, crt, hbd and thiL derived from Clostridium sp. were substituted with genes derived from E. coli, respectively, the resulting recombinant microorganisms were measured for butanol productivity.

### 2-1: Construction of pKKmhpFpaaFGHatoB Vector

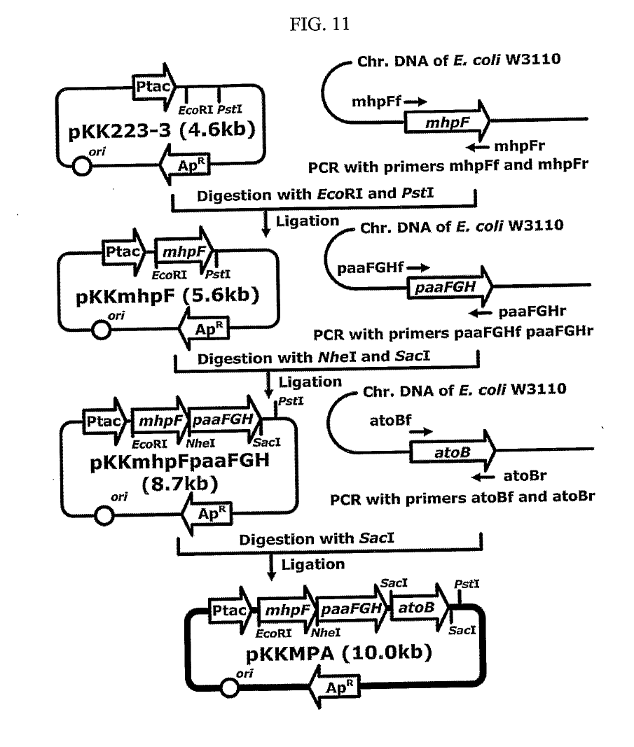

PCR was performed using primers of SEQ ID NOS: 33 to 38, with the chromosomal DNA of E. coli W3110 serving as a template, to amplify genes essential for the butanol biosynthesis pathway, including mhpF (coding for acetaldehyde dehydrogenase), paaFG (coding for enoyl-CoA hydratase), paaH (coding for 3-hydroxy-acyl-CoA dehydrogenase) and atoB (coding for acetyl-CoA acetyltransferase). These genes were sequentially cloned into a pKK223-3 expression vector (Pharmacia Biotech), thus constructed a novel recombinant expression vector, named pKKmhpFpaaFGHatoB (pKKMPA) (FIG. 11).

[SEQ ID NO: 33]

mhpFf:
5′-atgcgaattcatgagtaagcgtaaagtcgc-3′

[SEQ ID NO: 34]

mhpFr:
5′-tatcctgcaggagctctctagagctagcttaccgttcatgccgcttc
t-3′

[SEQ ID NO: 35]

paaFGHf:
5′-atacgctagcatgaactggccgcaggttat-3′

[SEQ ID NO: 36]

paaFGHr:
5′-tatcgagctcgccaggccttatgactcata-3′

[SEQ ID NO: 37]

atoBf:
5′-atacgagctctgcatcactgccctgctctt-3′

[SEQ ID NO: 38]

atoBr:
5′-tgtcgagctccgctatcgggtgtttttatt-3′

### 2-2: Construction of pTrc184bcdetfABbdhABgroESL Vector

PCR was performed using primers of SEQ ID NOS: 39 and 40, with the chromosomal DNA of Clostridium acetobutylicum serving as a template. The PCR product (etfAB) obtained was digested with KpnI and BamHI, followed by the insertion of the truncated PCR product into the pTrc184bcdbdhAB vector digested with the same restriction enzymes (KpnI and BamHI), thus constructed a novel recombinant expression vector, named pTrc184bcdetfABbdhAB (pTrc184BEB), which contain all of the bcd gene, the bdhAB gene and the etfAB gene.

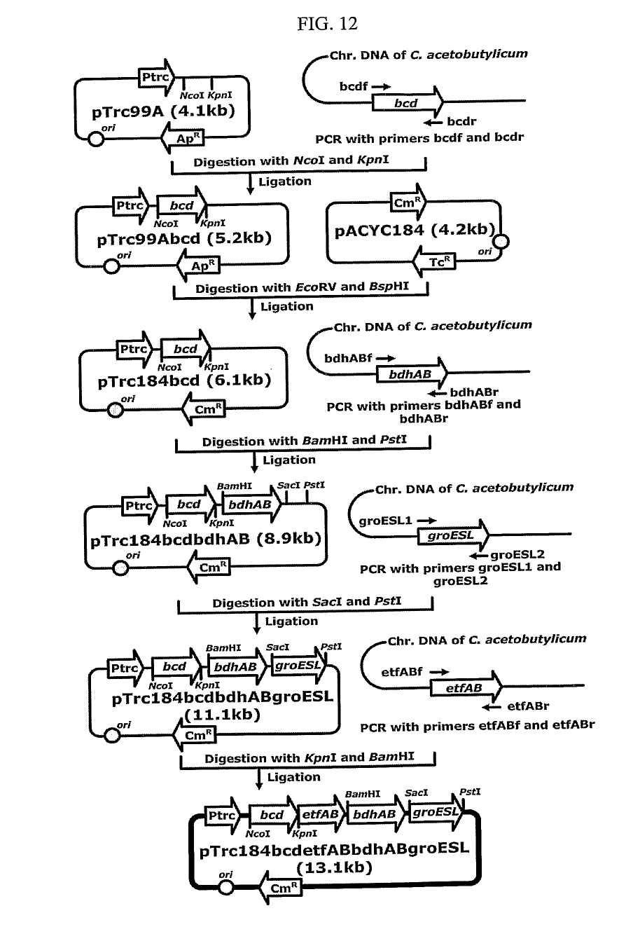

PCR was performed using primers of SEQ ID NOS: 41 and 42, with the chromosomal DNA of Clostridium acetobutylicum serving as a template. The PCR product obtained was digested with SacI and PstI, followed by the insertion of the truncated PCR product into the pTrc184bcdetfABbdhAB vector digested with the same restriction enzymes (SacI and PstI), thus constructed a novel recombinant expression vector, named pTrc184bcdetfABbdhABgroESL (pTrc184BEBG), which contain all of the bcd gene, the bdhAB gene, the etfAB gene and the groESL gene (FIG. 12).

[SEQ ID NO: 39]

| | |
|---|---|
| etfABf: | 5′-atacggtaccaaatgtagcaatggatgtaa-3′ |

[SEQ ID NO: 40]

| | |
|---|---|
| etfABr: | 5′-gtacggatcccttaattattagcagcttta-3′ |

[SEQ JD NO: 41]

| | |
|---|---|
| groESL1: | 5′-atgcgagctcaaaaagcgagaaaaaccata-3′ |

[SEQ ID NO: 42]

| | |
|---|---|
| groESL2: | 5′-gtacctgcagattagtacattccgcccatt-3′ |

### 2-3: Preparation of Butanol-Producing Microorganism

E. coli W3110 (WLLPA), lacking lacI, ldhA, pta and adhE, and E. coli W3110 (WLL) lacking lacI and ldhA, respectively prepared in Examples 1-1 and 1-2, were transformed with the pKKMPA vector of Example 3-1 and the pTrc184bcdbdhAB (pTrc184BB) vector of Example 1-10 or the pKKBEBG vector of Example 3-2, thus prepared recombinant mutant microorganisms capable of producing butanol (WLL+pKKMPA+pTrc184BB, WLLPA+pKKMPA+pTrc184BB, WLL+pKKMPA+pTrc184BEBG, and WLLPA+pKKMPA+pTrc184BEBG).

### 2-4 Assay for Butanol Productivity

The butanol-producing microorganisms prepared in Example 2-3 were cultured in the same manner as in Example 1-13 and measured for butanol productivity under the same conditions.

The results are summarized in Table 2, below. Compared to when only the butanol biosynthesis pathway of C. acetobutylicum was used, as shown in Table 2, butanol productivity was improved when E. coli-derived genes predicted to code the corresponding enzymes (adhE→mhpF, crt→paaFG, hbd→paaH, thiL→atoB) and the bcd and bdhAB genes derived from C. acetobutylicum were used in combination. That is, four (butyraldehyde dehydrogenase, crotonase, BHBD and THL) of the enzymes from Clostridium acetobutylicum essential for butanol production in E. coli can be substituted with enzymes encoded by mhpF, paaFG, paaH and atoB genes derived from E. coli, and these enzymes from E. coli were found to have higher activity than the corresponding enzymes from C. acetobutylicum, as demonstrated by the enhanced butanol production.

As demonstrated by the conspicuous increase in butanol productivity, the BCD enzyme, known to have poor activity in E. coli, was found to recover its activity with the expression of the co-factor encoding gene (etfAB) and the chaperone encoding gene (groESL).

TABLE 2

| | | Butanol |
|---|---|---|
| Strains | Containing genes | (mg/L) |
| WLL + pKKMPA + pTrc184BB | mhpF, paaFGH, atoB, | 18.4 |
| WLLPA + pKKMPA + pTrc184BB | bcd, bdhAB | 32.4 |
| WLL + pKKMPA + pTrc184BEBG | mhpF, paaFGH, atoB, | 365.5 |
| WLLPA + pKKMPA + pTrc184BEBG | bcd, bdhAB, etfAB, | 627.8 |
| | groESL | |

### INDUSTRIAL APPLICABILITY

As described in detail above, based on metabolic network reconstruction by gene deletion, metabolic engineering by amplification of desired genes and a method for increasing butyryl-CoA dehydrogenase activity, the present invention provides recombinant mutant microorganisms which have remarkably improved butanol productivity. Having advantages over Clostridium acetobutylicum in that they can be cultured easily and be further modified by manipulation of the metabolic pathways thereof, the recombinant mutant E. coli in accordance with the present invention is useful as a microorganism producing butanol for use in various industrial applications.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1.-21. (canceled)

22. A recombinant mutant microorganism having high butanol productivity, in which at least one selected from the group consisting of genes coding for enzymes involved in lactate biosynthesis, genes coding for enzymes involved in acetate biosynthesis, and genes coding for enzymes involved in ethanol biosynthesis is deleted or attenuated; and at least one gene coding for an enzyme involved in butanol biosynthesis is introduced or amplified.

23. The recombinant mutant microorganism having high butanol productivity according to claim 22, in which a lacI gene (coding for a lac operon repressor) is further deleted in the microorganism so as to enhance the expression of the gene coding for the enzyme involved in butanol biosynthesis.

24. The recombinant mutant microorganism having high butanol productivity according to claim 22, wherein said microorganism is selected from the group consisting of a bacterium, a yeast, and a fungus.

25. The recombinant mutant microorganism having high butanol productivity according to claim 24, wherein said bacterium is E. coli.

26. The recombinant mutant microorganism having high butanol productivity according to claim 22, wherein the gene coding for the enzyme involved in the lactate biosynthesis is ldhA (coding for lactate dehydrogenase).

27. The recombinant mutant microorganism having high butanol productivity according to claim 22, wherein the gene coding for the enzyme involved in the acetate biosynthesis is pta (coding for phosphoacetyltransferase).

28. The recombinant mutant microorganism having high butanol productivity according to claim 22, wherein the gene coding for the enzyme involved in the ethanol biosynthesis is adhE (coding for alcohol dehydrogenase).

29. The recombinant mutant microorganism having high butanol productivity according to claim 22, wherein the enzyme involved in butanol biosynthesis is at least one selected from the group consisting of thiolase (THL), 3-hydroxybutyryl-CoA dehydrogenase (BHBD), crotonase (CRO), butyryl-CoA dehydrogenase (BCD), butyraldehyde dehydrogenase (AAD), butanol dehydrogenase (BDH), and combinations thereof.

30. The recombinant mutant microorganism having high butanol productivity according to claim 29, wherein the THL is encoded by a gene selected from the group consisting of thl, thiL, phaA, and atoB.

31. The recombinant mutant microorganism having high butanol productivity according to claim 29, wherein the BCD is encoded by a bcd gene derived from Pseudomonas sp. or a ydbM gene derived from Bacillus sp.

32. The recombinant mutant microorganism having high butanol productivity according to claim 29, wherein the BCD is encoded by a bcd gene derived from Clostridium sp., and a chaperone-encoding gene is further introduced into the microorganism.

33. The recombinant mutant microorganism having high butanol productivity according to claim 32, in which a BCD co-factor-encoding gene (etfAB) is further introduced into the microorganism.

34. The recombinant mutant microorganism having high butanol productivity according to claim 32, wherein said chaperone-encoding gene is groESL.

35. The recombinant mutant microorganism having high butanol productivity according to claim 29, wherein the gene coding for the BHBD is a hbd gene derived from Clostridium sp. or a paaH gene derived from E. coli.

36. The recombinant mutant microorganism having high butanol productivity according to claim 29, wherein the gene coding for the CRO is a crt gene derived from Clostridium sp. or a paaFG gene derived from E. coli.

37. The recombinant mutant microorganism having high butanol productivity according to claim 29, wherein the gene coding for the AAD is an adhE gene derived from Clostridium sp. or a mhpF gene derived from E. coli.

38. The recombinant mutant microorganism having high butanol productivity according to claim 22, wherein the gene coding for the enzyme involved in the butanol biosynthesis is introduced into the microorganism by an expression vector containing a strong promoter.

39. The recombinant mutant microorganism having high butanol productivity according to claim 38, wherein the strong promoter is selected from the group consisting of a trc promoter, a tac promoter, a T7 promoter, a lac promoter and a trp promoter.

40. The recombinant mutant microorganism having high butanol productivity according to claim 39, wherein the expression vector containing the strong promoter further contains a gene coding for an enzyme selected from the group consisting of 3-hydroxybutyryl-CoA dehydrogenase, thiolase, butyraldehyde dehydrogenase, crotonase, butanol dehydrogenase, butyryl-CoA dehydrogenase and combinations thereof.

41. The recombinant mutant microorganism having high butanol productivity according to claim 40, wherein the expression vector further contains a chaperone-encoding gene and/or a BCD co-factor-encoding gene.

42. The recombinant mutant microorganism having high butanol productivity according to claim 40, wherein the expression vector is of any one selected from the group consisting of pKKHAT, pKKHAA, pKKHYAP, pKKHAP, pKKHPAP, pKKHKAP, and pKKMPA; and any one selected from the group consisting of pTrc184BBC and pTrc184BEBG.

43. A recombinant mutant microorganism having high butanol productivity, in which genes coding for enzymes involved in lactate biosynthesis, genes coding for enzymes involved in acetate biosynthesis, and genes coding for enzymes involved in ethanol biosynthesis are deleted or attenuated; and genes coding for thiolase (THL), 3-hydroxybutyryl-CoA dehydrogenase (BHBD), crotonase (CRO), butyryl-CoA dehydrogenase (BCD), butyraldehyde dehydrogenase (AAD), butanol dehydrogenase (BDH), a chaperone protein (groESL), and BCD co-factors (etfAB) are introduced or amplified.

44. A method for producing butanol, the method comprises culturing the recombinant mutant microorganism of claims 22, 29 or 32 to produce butanol; and recovering the butanol from the culture broth.

45. A method for producing butanol, the method comprises culturing the recombinant mutant microorganism of claim 43 to produce butanol; and recovering the butanol from the culture broth.


SEQUENCE LISTING (42 sequences)

SEQ ID NO: 1 - DNA, 72 bp, Artificial Sequence
  lacl_1st up primer
gtgaaaccagtaacgttatacgatgtcgcagagtatgccggtgtctcttagattgcagcattacacgtcttg

SEQ ID NO: 2 - DNA, 70 bp, Artificial Sequence
  lacl_1st do primer
tcactgcccgctttccagtcgggaaacctgtcgtgccagctgcattaatgcacttaacggctgacatggg

SEQ ID NO: 3 - DNA, 72 bp, Artificial Sequence
  ldhA1stup primer
atgaaactcgccgtttatagcacaaaacagtacgacaagaagtacctgcagattgcagcattacacgtcttg

SEQ ID NO: 4 - DNA, 71 bp, Artificial Sequence
  ldhA1stdo primer
ttaaaccagttcgttcgggcaggtttcgcctttttccagattgcttaagtcacttaacggctgacatggga

SEQ ID NO: 5 - DNA, 72 bp, Artificial Sequence
  pta1stup primer
gtgtcccgtattattatgctgatccctaccggaaccagcgtcggtctgacgattgcagcattacacgtcttg

SEQ ID NO: 6 - DNA, 69 bp, Artificial Sequence
  pta1stdo primer
ttactgctgctgtgcagactgaatcgcagtcagcgcgatggtgtagacgaacttaacggctgacatggg

SEQ ID NO: 7 - DNA, 72 bp, Artificial Sequence
  adhE1stup primer
cgtgaatatgccagtttcactcaagagcaagtagacaaaatcttccgcgcgattgcagcattacacgtcttg

SEQ ID NO: 8 - DNA, 70 bp, Artificial Sequence
  adhE1stdo primer
taatcacgaccgtagtaggtatccagcagaatctgtttcagctcggagatcacttaacggctgacatggg

SEQ ID NO: 9 - DNA, 30 bp, Artificial Sequence
  hbdf primer
acgcgaattcatgaaaaaggtatgtgttat

SEQ ID NO: 10 - DNA, 47 bp, Artificial Sequence
  hbdr primer
gcgtctgcaggagctcctgtctctagaatttgataatggggattctt

SEQ ID NO: 11 - DNA, 30 bp, Artificial Sequence
  adhEf primer
acgctctagatataaggcatcaaagtgtgt

SEQ ID NO: 12 - DNA, 30 bp, Artificial Sequence
  adhEr primer
gcgtgagctccatgaagctaatataatgaa

SEQ ID NO: 13 - DNA, 30 bp, Artificial Sequence
  thiLf primer
acgcgagctctatagaattggtaaggatat

SEQ ID NO: 14 - DNA, 30 bp, Artificial Sequence
  thiLr primer
gcgtgagctcattgaacctccttaataact

SEQ ID NO: 15 - DNA, 30 bp, Artificial Sequence
  atof primer
atacgagctctacggcgagcaatggatgaa

SEQ ID NO: 16 - DNA, 30 bp, Artificial Sequence
  ator primer
gtacgagctcgattaattcaaccgttcaat

SEQ ID NO: 17 - DNA, 39 bp, Artificial Sequence
  phaAf primer
agtcgagctcaggaaacagatgactgacgttgtcatcgt

SEQ ID NO: 18 - DNA, 30 bp, Artificial Sequence
  phaAr primer
atgcgagctcttatttgcgctcgactgcca

SEQ ID NO: 19 - DNA, 30 bp, Artificial Sequence
  ydbMf primer
agcttctagagatgggttacctgacatata

SEQ ID NO: 20 - DNA, 30 bp, Artificial Sequence
  ydbMr primer
agtctctagattatgactcaaacgcttcag

SEQ ID NO: 21 - DNA, 30 bp, Artificial Sequence
  bcdPA01f primer
agcttctagaactgctccttggacagcgcc

SEQ ID NO: 22 - DNA, 30 bp, Artificial Sequence
  bcdPA01r primer
agtctctagaggcaggcaggatcagaacca

SEQ ID NO: 23 - DNA, 30 bp, Artificial Sequence
  bcdKT2440f primer
agcttctagaactgttccttggacagcgcc

SEQ ID NO: 24 - DNA, 30 bp, Artificial Sequence; identical to SEQ ID NO: 22
  bcdKT2440r primer

SEQ ID NO: 25 - DNA, 30 bp, Artificial Sequence
  groESLf primer
agcttctagactcaagattaacgagtgcta

SEQ ID NO: 26 - DNA, 30 bp, Artificial Sequence
  groESLr primer
tagctctagattagtacattccgcccattc

SEQ ID NO: 27 - DNA, 26 bp, Artificial Sequence
  bcdf primer
agcgccatggattttaatttaacaag

SEQ ID NO: 28 - DNA, 30 bp, Artificial Sequence
  bcdr primer
agtcggtacccctccttaaattatctaaaa

SEQ ID NO: 29 - DNA, 30 bp, Artificial Sequence
  bdhABf primer
acgcggatccgtagtttgcatgaaatttcg

SEQ ID NO: 30 - DNA, 41 bp, Artificial Sequence
  bdhABr primer
agtcctgcagctatcgagctctataatggctacgcccaaac

SEQ ID NO: 31 - DNA, 30 bp, Artificial Sequence
  crtf primer
actcgagctcaaaagccgagattagtacgg

SEQ ID NO: 32 - DNA, 30 bp, Artificial Sequence
  crtr primer
gcgtctgcagcctatctatttttgaagcct

SEQ ID NO: 33 - DNA, 30 bp, Artificial Sequence
  mhpFf primer
atgcgaattcatgagtaagcgtaaagtcgc

SEQ ID NO: 34 - DNA, 48 bp, Artificial Sequence
  mhpFr primer
tatcctgcaggagctctctagagctagcttaccgttcatgccgcttct

SEQ ID NO: 35 - DNA, 30 bp, Artificial Sequence
  paaFGHf primer
atacgctagcatgaactggccgcaggttat

SEQ ID NO: 36 - DNA, 30 bp, Artificial Sequence
  paaFGHr primer
tatcgagctcgccaggccttatgactcata

SEQ ID NO: 37 - DNA, 30 bp, Artificial Sequence
  atoBf primer
atacgagctctgcatcactgccctgctctt

SEQ ID NO: 38 - DNA, 30 bp, Artificial Sequence
  atoBr primer
tgtcgagctccgctatcgggtgtttttatt

SEQ ID NO: 39 - DNA, 30 bp, Artificial Sequence
  atfABf primer
atacggtaccaaatgtagcaatggatgtaa

SEQ ID NO: 40 - DNA, 30 bp, Artificial Sequence
  etfABr primer
gtacggatcccttaattattagcagcttta

SEQ ID NO: 41 - DNA, 30 bp, Artificial Sequence
  groESL1 primer
atgcgagctcaaaaagcgagaaaaaccata

SEQ ID NO: 42 - DNA, 30 bp, Artificial Sequence
  groESL2 primer
gtacctgcagattagtacattccgcccatt